(19) United States
(12) Patent Application Publication
(10) Pub. No.: US 2025/0200837 A1
(43) Pub. Date: Jun. 19, 2025

(54) MEDICAL IMAGE PROCESSING APPARATUS AND MEDICAL IMAGE PROCESSING METHOD

(71) Applicant: CANON MEDICAL SYSTEMS CORPORATION, Otawara-shi (JP)

(72) Inventor: Shinsuke TSUKAGOSHI, Nasushiobara (JP)

(73) Assignee: CANON MEDICAL SYSTEMS CORPORATION, Otawara-shi (JP)

(21) Appl. No.: 18/973,219

(22) Filed: Dec. 9, 2024

(30) Foreign Application Priority Data
Dec. 13, 2023 (JP) 2023-210381

(51) Int. Cl. G06T 11/00; A61B 6/03; A61B 6/42

(52) U.S. Cl. CPC G06T 11/005; A61B 6/032; A61B 6/4241

(57) ABSTRACT

A medical image processing apparatus of an embodiment includes processing circuitry. The processing circuitry is configured to acquire imaging conditions including a tube voltage of an X-ray tube used at the time of capturing a past CT image of a subject, determine a range of energy bins to be used to generate a photon counting CT image corresponding to the past CT image among a plurality of energy bins set for counting X-ray photons detected by new imaging of the subject using a photon counting CT apparatus for each energy band based on the acquired imaging conditions, and generate the photon counting CT image using count values of X-ray photons in the determined range of energy bins.

### CROSS-REFERENCE TO RELATED APPLICATION

The present application claims priority based on Japanese Patent Application No. 2023-210381 filed Dec. 13, 2023, the content of which is incorporated herein by reference.

### FIELD

Embodiments disclosed in this specification and the drawings relate to a medical image processing apparatus and a medical image processing method.

### BACKGROUND

In follow-up examinations after treatment and regular health checkups, medical images captured in the past may be compared with newly captured medical images to observe changes in the condition of a subject. At the time of comparing such medical images, it is important to match imaging conditions for both images to eliminate differences in image appearance resulting from differences in the imaging conditions and make it easier to check changes in the condition of the subject.

In recent years, in the field of medical image diagnostic apparatuses, new photon counting CT apparatuses (Photon Counting Computed Tomography; PCCT) that use photon counting detectors, which are semiconductor detectors, have come to be used in addition to conventional CT (Computed Tomography) apparatuses that use detectors composed of scintillators and photodiodes. Photon counting CT apparatuses can create a variety of images (integral images (conventional images), Mono, k-edge, etc.).

When it is necessary to compare a medical image captured using a conventional CT apparatus with a new medical image captured using a photon counting CT apparatus, it is not easy to compare the two images at the same energy level. With a conventional CT apparatus, a tube voltage is changed arbitrarily depending on the subject to capture an image, but when capturing an image using a photon-counting CT apparatus, image-capturing is generally performed with the tube voltage of an X-ray tube set to 120 kVp or 140 kVp. For this reason, in a case where a past medical image was captured using a conventional CT apparatus with a tube voltage set to 80 kVp, even if a CT image (integral image) is created using combined data of count values of a plurality of energy bands (energy bins) collected by the photon counting CT apparatus, the appearance of the image will differ due to the difference in tube voltage, and the two images cannot be simply compared. Furthermore, even when medical images captured using a photon counting CT apparatus are compared, if the images were captured under different image-capturing conditions, such as with different energy bin settings, it is not easy to compare the two images at the same energy level.

### BRIEF DESCRIPTION OF THE DRAWINGS

FIG. 1 is a diagram showing an example of a configuration of a photon counting CT apparatus 1 according to a first embodiment.

FIG. 2 is a diagram showing an example of a configuration of a DAS 16 according to the first embodiment.

FIG. 3 is a diagram showing an example of imaging condition data D1 according to the first embodiment.

FIG. 4 is a flowchart showing an example of image generation processing performed by the photon counting CT apparatus 1 according to the first embodiment.

FIG. 5 is a flowchart showing an example of reconstruction condition determination processing performed by the photon counting CT apparatus 1 according to the first embodiment.

FIG. 6 is a diagram illustrating data used for reconstruction among count values for each energy bin in the photon counting CT apparatus 1 according to the first embodiment.

FIG. 7 is a flowchart showing an example of reconstruction condition determination processing performed by the photon counting CT apparatus 1 according to a second embodiment.

FIG. 8 is a graph showing a relationship between X-ray energy and X-ray intensity for each tube voltage according to the second embodiment.

FIG. 9 is a graph showing a relationship between X-ray energy and X-ray intensity for each tube current according to the second embodiment.

### DETAILED DESCRIPTION

Hereinafter, a medical image processing apparatus and a medical image processing method according to an embodiment will be described with reference to the drawings.

A medical image processing apparatus according to an embodiment includes processing circuitry. The processing circuitry is configured to acquire imaging conditions including a tube voltage of an X-ray tube used at the time of capturing a past CT image of a subject, determine a range of energy bins to be used to generate a photon counting CT image corresponding to the past CT image among a plurality of energy bins set for counting X-ray photons detected by new imaging of the subject using a photon counting CT apparatus for each energy band based on the acquired imaging conditions, and generate the photon counting CT image using count values of X-ray photons in the determined range of energy bins.

### First Embodiment

### [Configuration of Photon Counting CT Apparatus]

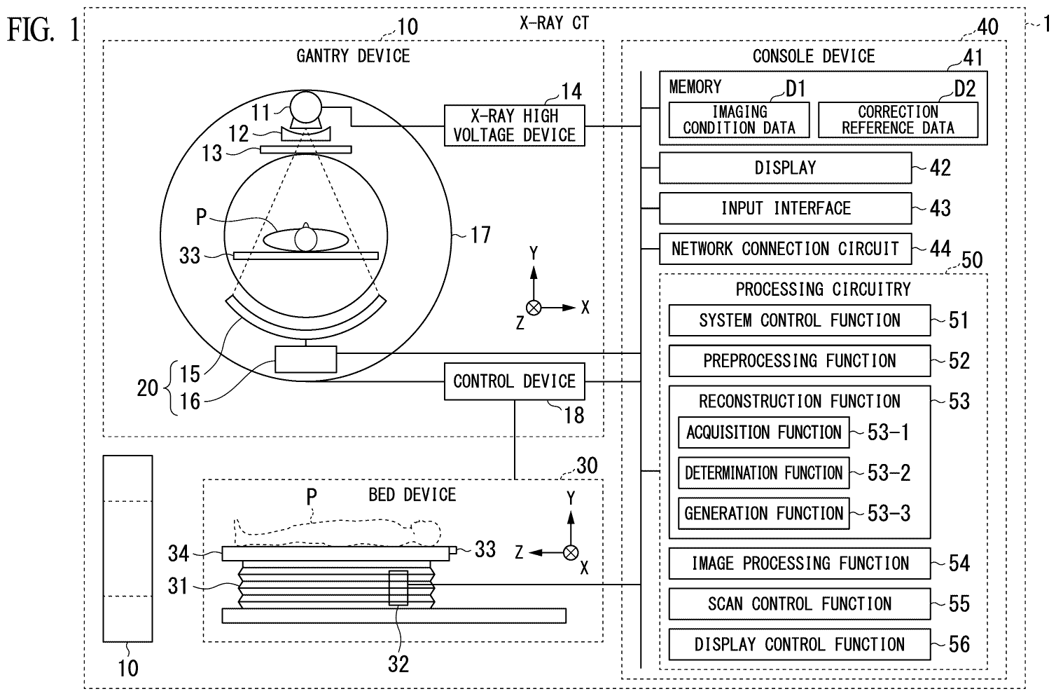

FIG. 1 is a diagram showing an example of a photon counting CT apparatus 1 according to a first embodiment. The photon counting CT apparatus 1 can generate image data in which a material of a test subject through which X-rays have passed is discriminated using a direct type detector such as a semiconductor detector with excellent energy resolution. Furthermore, the photon counting CT apparatus 1 can generate image data similar to conventional CT images by using combined data of count values in a plurality of energy bands (energy bins).

The photon counting CT apparatus 1 includes, for example, a gantry device 10, a bed device 30, and a console device 40. For convenience of explanation, FIG. 1 shows both a view of the gantry device 10 from a Z-axis direction and a view from an X-axis direction, but in reality, there is only one gantry device 10. In the present embodiment, a rotation axis of a rotating frame 17 in a non-tilted state or the longitudinal direction of a top plate 33 of the bed device 30 is defined as the Z-axis direction, an axis orthogonal to the Z-axis direction and horizontal to the floor surface is defined as the X-axis direction, and a direction orthogonal to the Z-axis direction and perpendicular to the floor surface is defined as a Y-axis direction.

### <Gantry Device 10>

The gantry device 10 includes, for example, an X-ray tube 11, a wedge 12, a collimator 13, an X-ray high voltage device 14, an X-ray detector 15, a data acquisition system (hereinafter, DAS) 16, the rotating frame 17, and a control device 18. The X-ray detector 15 and the DAS 16 constitute a detector module 20.

The X-ray tube 11 generates X-rays by radiating thermoelectrons from a cathode (filament) to an anode (target) when a high voltage is applied from the X-ray high voltage device 14. The X-ray tube 11 includes a vacuum tube. For example, the X-ray tube 11 is a rotating anode type X-ray tube that generates X-rays by radiating thermoelectrons to a rotating anode.

The wedge 12 is a filter for adjusting the amount of X-rays radiated from the X-ray tube 11 to a subject P. The wedge 12 attenuates X-rays that pass through the wedge 12 such that the distribution of the amount of X-rays radiated from the X-ray tube 11 to the subject P becomes a predetermined distribution. The wedge 12 is also called a wedge filter or a bow-tie filter. The wedge 12 is made by processing aluminum such that it has a predetermined target angle and a predetermined thickness, for example.

The collimator 13 is a mechanism for narrowing the radiation range of X-rays that have passed through the wedge 12. The collimator 13 narrows the radiation range of X-rays, for example, by forming a slit by combining a plurality of lead plates. The collimator 13 may also be called an X-ray aperture. The narrowing range of the collimator 13 may be mechanically operable.

The X-ray high voltage device 14 includes, for example, a high voltage generator that is not shown and an X-ray control device that is not shown. The high voltage generator has an electric circuit including a transformer, a rectifier, and the like and generates a high voltage to be applied to the X-ray tube 11. The X-ray control device controls the output voltage of the high voltage generator in response to the amount of X-rays to be generated by the X-ray tube 11. The high voltage generator may boost the voltage using the above-mentioned transformer or boost the voltage using an inverter. The X-ray high voltage device 14 may be provided on the rotating frame 17 or on the side of a fixed frame (not shown) of the gantry device 10.

The X-ray detector 15 detects the intensity of X-rays generated by the X-ray tube 11 and passing through the subject P. The X-ray detector 15 outputs an electrical signal (which may be an optical signal or the like) in accordance with the detected intensity of X-rays to the DAS 16. The X-ray detector 15 has, for example, a plurality of X-ray detection element rows. Each of the plurality of X-ray detection element rows has a plurality of X-ray detection elements arranged in a channel direction along an arc having the focal point of the X-ray tube 11 as a center. The plurality of X-ray detection element rows are arranged in a slice direction (row direction).

The X-ray detector 15 is, for example, a direct detection type detector. For example, a semiconductor diode with electrodes attached to both ends of a semiconductor can be applied as the X-ray detector 15. X-ray photons incident on the semiconductor are converted into electron-hole pairs. The number of electron-hole pairs generated by incidence of one X-ray photon depends on the energy of the incident X-ray photons. Electrons and holes are respectively attracted to a pair of electrodes formed on both ends of the semiconductor. The pair of electrodes generates an electrical pulse having a pulse height value according to the charge of the electron-hole pairs. One electrical pulse has a pulse height value according to the energy of the incident X-ray photons.

The DAS 16 collects count data (count values) indicating the number of counts of X-ray photons detected by X-ray detector 15 with respect to a plurality of energy bins, for example, according to a control signal from the control device 18. Count values with respect to a plurality of energy bins correspond to an energy spectrum of X-rays incident on the X-ray detector 15, modified according to the response characteristics of the X-ray detector 15. The DAS 16 outputs detection data based on digital signals to the console device 40. The detection data is a digital value of a count value identified by a channel number and a row number of an X-ray detection element that is a generation source, and a view number indicating a collected view. A view number is a number that changes according to rotation of rotating frame 17 and, for example, a number that is incremented according to rotation of rotating frame 17. Therefore, the view number is information indicating a rotation angle of the X-ray tube 11. A view period is a period that falls between a rotation angle corresponding to a certain view number and a rotation angle corresponding to the next view number.

The DAS 16 may detect a view change by a timing signal input from the control device 18, by an internal timer, or by a signal obtained from a sensor that is not shown. When X-rays are continuously emitted by the X-ray tube 11 during full scanning, the DAS 16 collects a group of detection data for the entire circumference (360 degrees). When X-rays are continuously emitted by the X-ray tube 11 during half scanning, the DAS 16 collects detection data for half the circumference (180 degrees+fan angle). The DAS 16 processes detection data detected by the semiconductor detector.

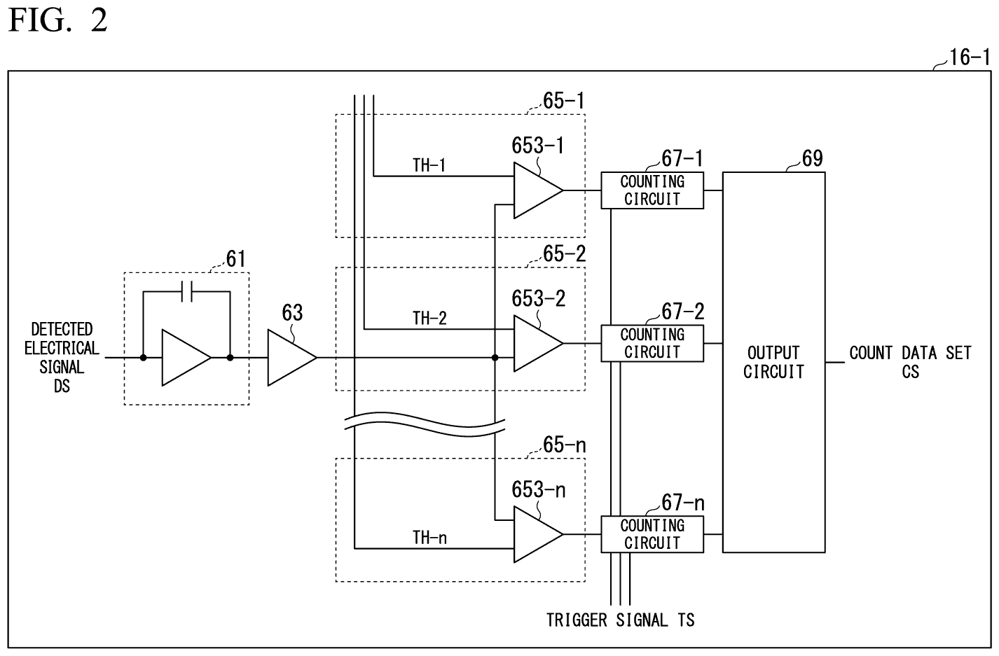

FIG. 2 is a diagram showing an example of a configuration of the DAS 16 according to the first embodiment. The DAS 16 has as many readout channels as the number of channels corresponding to the number of X-ray detection elements. These readout channels are implemented in parallel on an integrated circuit such as an ASIC. FIG. 2 shows only the configuration of a DAS 16-1 for one readout channel.

The DAS 16-1 includes a preamplifier circuit 61, a waveform shaping circuit 63, a plurality of pulse height discrimination circuits 65, a plurality of counting circuits 67, and an output circuit 69. The preamplifier circuit 61 amplifies a detected electrical signal DS (current signal) from a connected X-ray detection element. For example, the preamplifier circuit 61 converts the current signal from the connected X-ray detection element into a voltage signal having a voltage value (pulse height value) proportional to the charge amount of the current signal. The waveform shaping circuit 63 is connected to the preamplifier circuit 61. The waveform shaping circuit 63 shapes the waveform of the voltage signal from the preamplifier circuit 61. For example, the waveform shaping circuit 63 reduces the pulse width of the voltage signal from the preamplifier circuit 61. The waveform shaping circuit 63 is connected to a plurality of counting channels corresponding to the number of energy bands (energy bins). When n energy bins are set, the waveform shaping circuit 63 is provided with n counting channels. Each counting channel has a pulse height discrimination circuit 65-n and a counting circuit 67-n.

Each pulse height discrimination circuits 65-n discriminates the energy of X-ray photons detected by the X-ray detection element, which is the pulse height value of the voltage signal from the waveform shaping circuit 63. For example, the pulse height discrimination circuit 65-n has a comparison circuit 653-n. The voltage signal from the waveform shaping circuit 63 is input to one input terminal of each comparison circuits 653-n. A reference signal TH (reference voltage value) corresponding to a different threshold value is supplied from the control device 18 to the other input terminal of each comparison circuits 653-n.

For example, a reference signal TH-1 is supplied to the comparison circuit 653-1 for energy bin B1, a reference signal TH-2 is supplied to the comparison circuit 653-2 for energy bin B2, and a reference signal TH-n is supplied to the comparison circuit 653-n for energy bin Bn. Each reference signal TH has an upper limit reference value and a lower limit reference value. Each comparison circuit 653-n outputs an electrical pulse signal when the voltage signal from the waveform shaping circuit 63 has a pulse height value corresponding to the energy bin corresponding to each reference signal TH. For example, the comparison circuit 653-1 outputs an electrical pulse signal when the pulse height value of the voltage signal from the waveform shaping circuit 63 is a pulse height value corresponding to energy bin B1 (when it falls between the reference signals TH-1 and TH-2). On the other hand, the comparison circuit 653-1 for energy bin B1 does not output an electrical pulse signal when the pulse height value of the voltage signal from the waveform shaping circuit 63 is not a pulse height value corresponding to energy bin B1. Further, for example, the comparison circuit 653-2 outputs an electrical pulse signal when the pulse height value of the voltage signal from the waveform shaping circuit 63 is a pulse height value corresponding to energy bin B2 (when it falls between the reference signals TH-2 and TH-3).

The counting circuit 67-n counts electrical pulse signals from the pulse height discrimination circuit 65-n at a readout period that coincides with a view switching period. For example, a trigger signal TS is supplied to the counting circuit 67-n from the control device 18 at the switching timing of each view. When the trigger signal TS is supplied, the counting circuit 67-n adds 1 to a count number stored in an internal memory each time an electrical pulse signal is input from the pulse height discrimination circuit 65-n. When the next trigger signal is supplied, the counting circuit 67-n reads out data of the count number (i.e., a count value) stored in the internal memory and supplies the same to the output circuit 69. Further, the counting circuit 67-n resets the count number stored in the internal memory to an initial value each time the trigger signal TS is supplied. In this manner, the counting circuit 67-n counts a count number for each view.

The output circuit 69 is connected to counting circuits 67-n for a plurality of readout channels mounted on the X-ray detector 15. The output circuit 69 integrates count values from the counting circuits 67-n for the plurality of readout channels for each of a plurality of energy bins to generate count values for a plurality of readout channels for each view. The count values for each energy bin are a set of data of count numbers defined by a channel, a segment (row), and an energy bin. The count values for each energy bin are transmitted to the console device 40 for each view. The count values for each view are called a count data set CS. Furthermore, the output circuit 69 transmits data detected for each pixel detected by the X-ray detector 15 to the console device 40. The detection data includes at least one of the data detected for each pixel and the count values for each energy bin.

Referring back to FIG. 1, the rotating frame 17 is an annular member that supports the X-ray tube 11, the wedge 12, the collimator 13, and the X-ray detector 15 such that the X-ray tube 11, the wedge 12, and the collimator 13 face the X-ray detector 15. The rotating frame 17 is supported by a fixed frame such that it is freely rotatable around the subject P introduced therein. The rotating frame 17 also supports the DAS 16. Detection data output by the DAS 16 is transmitted through optical communication from a transmitter having a light-emitting diode (LED) provided on the rotating frame 17 to a receiver having a photodiode provided on a non-rotating part (e.g., the fixed frame) of the gantry device 10, and is transferred to the console device 40 by the receiver. Note that the method of transmitting the detection data from the rotating frame 17 to the non-rotating part is not limited to the above-mentioned method using optical communication, and any non-contact type transmission method may be adopted. The rotating frame 17 is not limited to an annular member, and may be an arm-like member as long as it can support and rotate the X-ray tube 11 and the like.

The photon counting CT apparatus 1 is, for example, a rotate/rotate-type X-ray CT apparatus (third generation CT) in which both the X-ray tube 11 and the X-ray detector 15 are supported by the rotating frame 17 and rotate around the subject P, but is not limited thereto and may be a stationary/rotate-type X-ray CT apparatus (fourth generation CT) in which a plurality of X-ray detection elements arranged in a circular ring are fixed to a fixed frame and the X-ray tube 11 rotates around the subject P.

The control device 18 includes processing circuitry having a processor such as a central processing unit (CPU), for example. The control device 18 receives an input signal from the console device 40 or an input interface attached to the gantry device 10, and controls the operations of the gantry device 10, the bed device 30, and the DAS 16. For example, the control device 18 rotates the rotating frame 17 and tilts the gantry device 10. When tilting the gantry device 10, the control device 18 rotates the rotating frame 17 around an axis parallel to the Z-axis direction on the basis of an inclination angle (tilt angle) input to the input interface. The control device 18 ascertains the rotation angle of the rotating frame 17 from the output of a sensor that is not shown, or the like. The control device 18 also controls the energy bins (reference signals TH) of the DAS 16. The control device 18 may be provided in the gantry device 10 or in the console device 40.

### <Bed Device 30>

The bed device 30 is a device that loads and moves the subject P to be scanned and introduces the subject P into the rotating frame 17 of the gantry device 10. The bed device 30 includes, for example, a base 31, a bed drive device 32, the top plate 33, and a support frame 34. The base 31 includes a housing that supports the support frame 34 such that the support frame 34 can move in the vertical direction (Y-axis direction). The bed drive device 32 includes a motor and an actuator. The bed drive device 32 moves the top plate 33 along the support frame 34 in the longitudinal direction (Z-axis direction) of the top plate 33. The bed drive device 32 also moves the top plate 33 in the vertical direction (Y-axis direction). The top plate 33 is a plate-shaped member on which the subject P is placed.

The bed drive device 32 may move not only the top plate 33 but also the support frame 34 in the longitudinal direction of the top plate 33. On the contrary, the gantry device 10 may be movable in the Z-axis direction, and the movement of the gantry device 10 may be controlled such that the rotating frame 17 comes around the subject P. Alternatively, both the gantry device 10 and the top plate 33 may be configured to be movable. Further, the photon counting CT apparatus 1 may be an apparatus in which the subject P is scanned in a standing or sitting position. In this case, the photon counting CT apparatus 1 has a subject support mechanism instead of the bed device 30, and the gantry device 10 rotates the rotating frame 17 around an axial direction perpendicular to the floor surface.

### <Console Device 40>

The console device 40 includes, for example, a memory 41, a display 42, an input interface 43, a network connection circuit 44, and processing circuitry 50. Although the console device 40 is described as being separate from the gantry device 10 in the present embodiment, the gantry device 10 may include some or all of the components of the console device 40.

The memory 41 is realized, for example, by a semiconductor memory element such as a random access memory (RAM), a flash memory, a hard disk, an optical disc, or the like. The memory 41 stores, for example, detection data, projection data, reconstructed image data (photon counting CT image data), information on the subject P, imaging condition data D1, correction reference data D2, and the like. The memory 41 stores, for example, count values with respect to a plurality of energy bins transmitted from the gantry device 10. Such data may be stored in an external memory with which the photon counting CT apparatus 1 can communicate, instead of (or in addition to) the memory 41. The external memory is controlled by a cloud server that manages the external memory, for example, by receiving a read/write request.

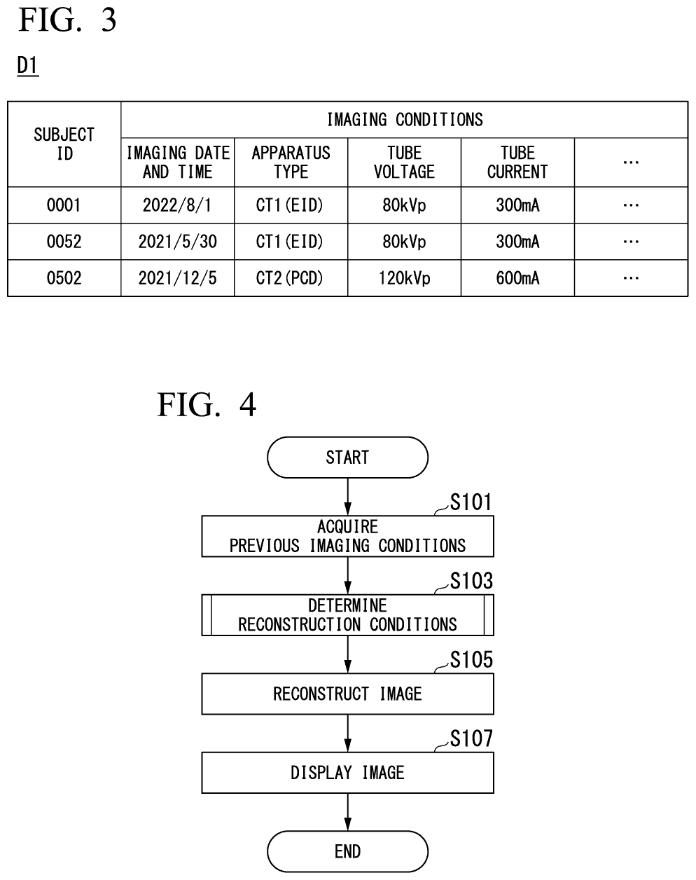

The imaging condition data D1 includes information on imaging conditions used when a past CT image of the subject was captured. The imaging condition data D1 is used, for example, to determine a range of energy bins to be used to generate a new photon counting CT image corresponding to the past CT image, among a plurality of energy bins set in the photon counting CT apparatus 1. For example, the imaging condition data D1 is included in an image examination order transmitted from a radiology information system (hereinafter, RIS), which is a computer system that supports operations in an imaging diagnosis department, and is stored in the memory 41 of the console device 40. Alternatively, the imaging condition data D1 is imaging conditions used in past imaging using the photon counting CT apparatus 1 that are stored in the memory 41 of the console device 40. FIG. 3 is a diagram showing an example of the imaging condition data D1 according to the first embodiment. As shown in FIG. 3, in the imaging condition data D1, imaging conditions such as “imaging date and time,” “apparatus type (conventional CT apparatus/photon counting CT apparatus),” “tube voltage,” and “tube current” are registered in association with a “subject ID” for identifying a subject.

Referring back to FIG. 1, the display 42 displays various types of information. For example, the display 42 displays medical images (CT images) generated by the processing circuitry, graphical user interface (GUI) images for receiving various operations performed by an operator such as a doctor or an engineer, and the like. The display 42 is, for example, a liquid crystal display, a cathode ray tube (CRT), an organic electroluminescence (EL) display, and the like. The display 42 may be provided on the gantry device 10. The display 42 may be a desktop type, or a display device (e.g., a tablet terminal) capable of wireless communication with the main body of the console device 40. The display 42 is an example of a “display device.”

The input interface 43 receives various input operations performed by an operator and outputs an electrical signal indicating the content of the received input operations to the processing circuitry 50. For example, the input interface 43 receives input operations such as collection conditions at the time of collecting detection data or projection data, reconstruction conditions at the time of reconstructing a CT image, image processing conditions at the time of generating a post-processed image from a CT image, and energy bin setting conditions. For example, the input interface 43 is realized by a mouse, a keyboard, a touch panel, a track ball, a switch, a button, a joystick, a camera, an infrared sensor, a microphone, or the like.

The input interface 43 may be provided in the gantry device 10. The input interface 43 may also be realized by a display device (e.g., a tablet terminal) capable of wireless communication with the main body of the console device 40. Note that in this specification, the input interface is not limited to one equipped with physical operating parts such as a mouse and a keyboard. For example, examples of the input interface also include electrical signal processing circuitry that receives an electrical signal corresponding to an input operation from external input equipment provided separately from the apparatus and outputs this electrical signal to a control circuit.

The network connection circuit 44 includes, for example, a network card having a printed circuit board, a wireless communication module, or the like. The network connection circuit 44 implements an information communication protocol depending on the type of a network to be connected.

The processing circuitry 50 controls the overall operation of the photon counting CT apparatus 1, the operation of the gantry device 10, and the operation of the bed device 30. The processing circuitry 50 executes, for example, a system control function 51, a preprocessing function 52, a reconstruction function 53, an image processing function 54, a scan control function 55, a display control function 56, and the like. The reconstruction function 53 includes, for example, an acquisition function 53-1, a determination function 53-2, a generation function 53-3, and the like. These components are realized, for example, by a hardware processor (computer) executing a program (software) stored in the memory 41. The hardware processor means, for example, circuitry such as a CPU, a graphics processing unit (GPU), an ASIC, a programmable logic device (for example, a simple programmable logic device (SPLD), a complex programmable logic device (CPLD), or a field programmable gate array (FPGA)).

Instead of storing the program in the memory 41, the program may be directly built into the circuit of the hardware processor. In this case, the hardware processor realizes the function by reading and executing the program built into the circuit. The hardware processor is not limited to being configured as a single circuit, but may be configured as a single hardware processor by combining a plurality of independent circuits to realize each function. In addition, a plurality of components may be integrated into a single hardware processor to realize each function.

Each component of the console device 40 or the processing circuitry 50 may be distributed and realized by a plurality of pieces of hardware. The processing circuitry 50 may be realized by a processing device capable of communicating with the console device 40, rather than being a component included in the console device 40. The processing device is, for example, a workstation connected to a single photon counting CT apparatus, or a device (for example, a cloud server) connected to a plurality of photon counting CT apparatuses and collectively executing processing equivalent to that of the processing circuitry 50 which will be described below. The photon counting CT apparatus 1, the console device 40, the workstation, the cloud server, or a combination thereof is an example of a “medical information processing apparatus.”

The system control function 51 controls various functions of the processing circuitry 50 on the basis of an input operation received by the input interface 43. For example, the system control function 51 performs setting of energy bins, setting of a tube voltage and a tube current of the X-ray tube 11, and the like. The system control function 51 outputs setting conditions of the set energy bins, and the like to the control device 18.

The preprocessing function 52 performs preprocessing such as offset correction processing, inter-channel sensitivity correction processing, and beam hardening correction on detection data output by the DAS 16.

The reconstruction function 53 reconstructs a photon counting CT image of the subject P on the basis of detection data (count values). The reconstruction function 53 calculates an X-ray absorption amount for each of a plurality of reference materials on the basis of count values with respect to a plurality of energy bins, the energy spectrum of X-rays incident on the subject P, and a response function representing detector response characteristics stored in the memory 41. Processing of obtaining the X-ray absorption amount for each reference material in this manner is also called material discrimination. Reference materials can be set to any material, such as calcium, calcification, bone, fat, muscle, air, organs, lesions, hard tissue, soft tissue, and contrast material. The reconstruction function 53 reconstructs a photon counting CT image expressing a spatial distribution of a reference material to be imaged among the plurality of reference materials on the basis of the calculated X-ray absorption amount for each of the plurality of reference materials, and stores the generated CT image data in the memory 41.

The acquisition function 53-1 acquires imaging conditions including the tube voltage of the X-ray tube used at the time of capturing a past CT image of the subject. The acquisition function 53-1 acquires, as imaging conditions, settings of the wedge 12 and energy bins, and the like in addition to the tube voltage of the X-ray tube. The acquisition function 53-1 acquires, for example, imaging conditions (imaging condition data D1) from the memory 41. The acquisition function 53-1 is an example of an “acquisition unit.”

The determination function 53-2 determines a range of energy bins to be used to generate a photon counting CT image corresponding to a past CT image, among a plurality of energy bins set for counting X-ray photons for each energy band detected by new imaging of the subject using the photon counting CT apparatus 1 on the basis of the acquired imaging conditions. The past CT image is an image captured by a conventional CT apparatus. The past CT image may be an integral image captured by a photon counting CT apparatus. The determination function 53-2 is an example of a “determination unit.”

The generation function 53-3 generates a photon counting CT image using count values in the determined range of energy bins. The generation function 53-3 is an example of a “generation unit.”

The image processing function 54 converts CT image data into three-dimensional image data or cross-sectional image data with an arbitrary cross section using a known method on the basis of an input operation received by the input interface 43. Conversion to three-dimensional image data may be performed by the preprocessing function 52.

The scan control function 55 controls detection data collection processing in the gantry device 10 by issuing instructions to the X-ray high voltage device 14, the DAS 16, the control device 18, and the bed drive device 32. The scan control function 55 controls the operation of each unit when capturing positioning images and when capturing images to be used for diagnosis.

The display control function 56 displays, on the display 42, medical images (photon counting CT images) generated by the processing circuitry, GUI images for receiving various operations performed by an operator such as a doctor or an engineer. The display control function 56 displays a photon counting CT image corresponding to a past CT image of the subject, generated by the generation function 53-3, on the display 42. The display control function 56 is an example of a “display control unit.”

With the above configuration, the photon counting CT apparatus 1 scans the subject P in a scan mode such as helical scanning, conventional scanning, and step-and-shoot. Helical scanning is a mode in which the rotating frame 17 is rotated while the top plate 33 is moved to scan the subject P in a spiral shape. Conventional scanning is a mode in which the rotating frame 17 is rotated while the top plate 33 is stationary to scan the subject P in a circular orbit. Step-and-shoot is a mode in which the position of the top plate 33 is moved at regular intervals to perform conventional scanning in a plurality of scan areas.

### [Processing Flow]

Next, a series of flows of image generation processing of the photon counting CT apparatus 1 will be described, focusing on processing of the console device 40. FIG. 4 is a flowchart showing an example of image generation processing performed by the photon counting CT apparatus 1 according to the first embodiment. The image generation processing shown in FIG. 4 is started when the operator inputs an instruction to generate a photon counting CT image via the input interface 43, for example, after scanning the subject P.

First, the acquisition function 53-1 acquires imaging conditions used when a past (previous) CT image of the subject P was captured from imaging condition data D1 stored in the memory 41 (step S101). The imaging conditions include at least a tube voltage (kVp) of the X-ray tube used when the past CT image of the subject P was captured. In the following, an example of a case in which imaging conditions of “apparatus type (CT1 (EID))” and “tube voltage (80 kVp)” associated with a subject ID (“0001”) are acquired from the imaging condition data D1 shown in FIG. 3 will be described.

If the past CT image of the subject P is accompanied by information in a format conforming to the Digital Imaging and Communication in Medicine (DICOM) standard (hereinafter also referred to as a “DICOM tag”), the acquisition function 53-1 may acquire the imaging conditions from the information of this DICOM tag. Further, if the past CT image contains information (a character string or the like) indicating the imaging conditions, the acquisition function 53-1 may acquire the imaging conditions through image analysis of the past CT image. Further, if the past CT image cannot be acquired, the acquisition function 53-1 may acquire a tube voltage and the like input by the operator via the input interface 43 as the imaging conditions.

Next, the determination function 53-2 determines a range of energy bins to be used to generate a new photon counting CT image corresponding to the past CT image of the subject P among a plurality of energy bins set in the photon counting CT apparatus 1 on the basis of the acquired imaging conditions (step S103).

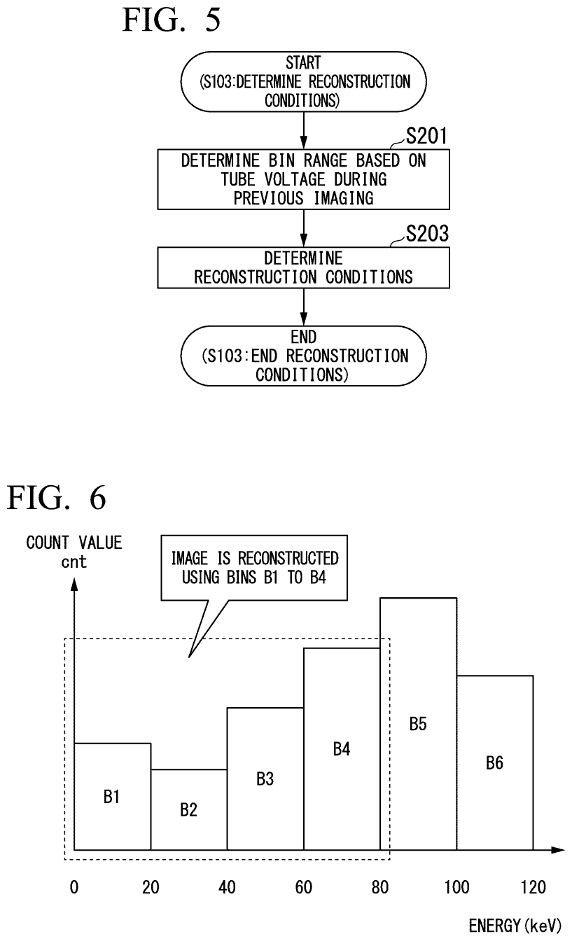

Hereinafter, details of processing of determining reconstruction conditions by the determination function 53-2 (step S103) will be specifically described. FIG. 5 is a flowchart showing an example of processing of determining reconstruction conditions by the photon counting CT apparatus 1 according to the first embodiment. FIG. 6 is a diagram illustrating data used for reconstruction among count values for each energy bin by the photon counting CT apparatus 1 according to the first embodiment.

First, the determination function 53-2 determines a range of energy bins on the basis of acquired past imaging conditions (tube voltage at the time of previous imaging) (step S201). For example, it is assumed that the tube voltage, which is the acquired past imaging condition, is “80 kVp” and that count values of X-ray photons for six energy bins B1 to B6 (step: 20 keV) have been obtained by new imaging of the subject P using the photon counting CT apparatus 1. The energy band of the energy bin B1 is 0 to 20 keV, the energy band of the energy bin B2 is 20 to 40 keV, the energy band of the energy bin B3 is 40 to 60 keV, the energy band of the energy bin B4 is 60 to 80 keV, the energy band of the energy bin B5 is 80 to 100 keV, and the energy band of the energy bin B6 is 100 to 120 keV. In this case, as shown in FIG. 6, the determination function 53-2 determines the range of the energy bins B1 to B4 (i.e., the energy band of 0 to 80 keV) close to the condition of the tube voltage value “80 kVp” which is the acquired past imaging condition as the range of energy bins to be used to generate a photon counting CT image. Next, the determination function 53-2 determines reconstruction conditions including the conditions of the determined range of energy bins (step S203).

That is, the determination function 53-2 determines the range of energy bins from the energy bin having the lowest energy band to the energy bin having the energy band corresponding to the tube voltage value included in the imaging conditions, among the plurality of energy bins, as the range of energy bins.

If the acquired past imaging conditions (tube voltage at the time of previous imaging) do not match the energy band divisions of the energy bins (boundary values between energy bins) of the photon counting CT apparatus 1, the determination function 53-2 determines an energy range up to an energy bin division close to the acquired past imaging conditions (tube voltage at the time of previous imaging) as an energy range. For example, if the tube voltage at the time of previous imaging in the past imaging conditions is “95 kVp,” the determination function 53-2 determines the range of energy bins (energy bins B1 to B5) up to “100 keV,” which is closest to the numerical value of this tube voltage “95 kVp,” as the range of energy bins to be used to generate a photon counting CT image.

That is, the determination function 53-2 determines the range of energy bins from the energy bin having the lowest energy band among the plurality of energy bins to the boundary value between energy bins that is closest to the tube voltage value included in the imaging conditions as the range of energy bins.

Alternatively, the determination function 53-2 may determine the range of energy bins to be used to generate a photon counting CT image by distinguishing between all energy bins whose upper limit values are equal to or less than the tube voltage at the time of previous imaging in the past imaging conditions, and energy bins having the energy band including the tube voltage at the time of previous imaging. For example, if the tube voltage at the time of previous imaging in the past imaging conditions is “95 kVp,” the determination function 53-2 determines the range of energy bins to be used to generate a photon counting CT image by distinguishing between energy bins B1 to B4 whose upper limit values are equal to or less than this tube voltage “95 kVp” and energy bin B5 including this tube voltage “95 kVp.”. In this case, in subsequent processing, the generation function 53-3 may generate a photon counting CT image using count values of the energy bins B1 to B4 and some of count values of the energy bin B5 taking into account the arithmetic average with a proportion from the lower limit value 80 keV in the energy band of the energy bin B5. The proportion of some of the count values of the energy bin B5 taking into account the arithmetic average is calculated, for example, by (95 keV (tube voltage at the time of previous imaging in past imaging conditions)−80 keV (lower limit value of the energy bin B5))/20 keV (step)=0.75.

That is, the determination function 53-2 determines the range of energy bins from the energy bin having the lowest energy band among the plurality of energy bins to the upper energy bin whose energy band includes the tube voltage value included in the imaging conditions as the range of energy bins, and the generation function 53-3 adjusts count values used to generate a photon counting CT image on the basis of the difference between the lower limit value of an energy band of upper energy bins and the tube voltage value included in the imaging conditions with respect to count values of the upper energy bins among the count values in the determined range of energy bins.

Alternatively, energy bin settings of the photon counting CT apparatus 1 may be changed on the basis of the tube voltage at the time of previous imaging, rescanning may be performed, and count values for the changed energy bin settings may be collected to generate a photon counting CT image corresponding to a past CT image.

The determination function 53-2 may determine the range of energy bins on the basis of instructions input by the operator via the input interface 43.

Referring back to FIG. 4, the generation function 53-3 reconstructs a photon counting CT image on the basis of the determined reconstruction conditions (using the count values in the determined range of energy bins) (step S105). Here, in order to generate a photon counting CT image corresponding to the past CT image (a CT generated by a conventional CT apparatus), the generation function 53-3 reconstructs a photon counting CT image (counting image) on the basis of combined data of count values in the energy band of 0 to 80 keV (data obtained by summing the count values of energy bins B1 to B4). The generation function 53-3 may reconstruct a plurality of photon counting CT images corresponding to the respective energy bins B1, B2, B3, and B4, and then perform arithmetic averaging of the images to create a photon counting CT image corresponding to the energy band of 0 to 80 keV.

Next, the display control function 56 causes the display 42 to display the photon counting CT image corresponding to the past CT image of the subject, which has been generated by the generation function 53-3 (step S107). At this time, the display control function 56 may cause the display 42 to display both the newly generated photon counting CT image and the past CT image such that they can be compared. Accordingly, the processing of this flowchart ends.

According to the first embodiment described above, it is possible to make it possible to compare medical images captured under different imaging conditions in a CT apparatus at the same energy level. Furthermore, by making such comparison of medical images possible, it is possible to support medical examinations by doctors. Moreover, since data in a wide energy range (e.g., 0 to 120 keV) can be obtained in new imaging using the photon counting CT apparatus, it is also possible to generate a spectral image unique to the photon counting CT apparatus.

### Second Embodiment

Next, the second embodiment will be described. The intensity and energy of X-rays (X-ray energy spectrum) output from the X-ray tube 11 change depending on the tube voltage of the X-ray tube 11. Therefore, if a new photon counting CT image is generated on the basis of only a difference in the tube voltage without considering such a difference in the X-ray energy spectrum, the image will appear different from a past CT image (reconstruction conditions will be different), and it may be difficult to compare the two images. Therefore, in a photon counting CT apparatus 1 of the second embodiment, a photon counting CT image is reconstructed in consideration of the energy spectrum of X-rays output from the X-ray tube 11 or the energy spectrum of X-rays incident on the X-ray detector 15. In the following description, the same components and functions as those in the first embodiment are denoted by the same reference numerals as those in the first embodiment, and detailed description thereof will be omitted.

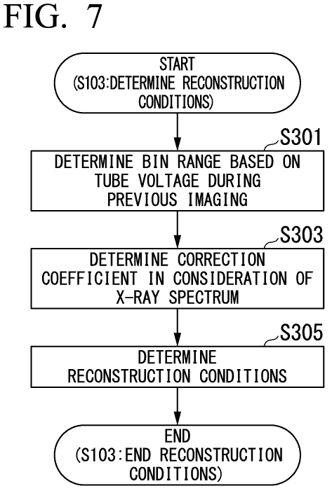

FIG. 7 is a flowchart showing an example of processing (step S103) of determining reconstruction conditions by the photon counting CT apparatus 1 according to the second embodiment. First, the determination function 53-2 determines a range of energy bins on the basis of acquired past imaging conditions (tube voltage at the time of previous imaging) (step S301). For example, it is assumed that the tube voltage is “80 kVp” as a past imaging condition, and count values for six energy bins B1 to B6 (step: 20 keV) are obtained by new imaging of the subject P using the photon counting CT apparatus 1. In this case, the determination function 53-2 determines the range of energy bins B1 to B4 (i.e., energy band of 0 to 80 keV) close to the condition of the tube voltage (80 kVp), which is the acquired past imaging condition, as the range of energy bins to be used to generate a photon counting CT image.

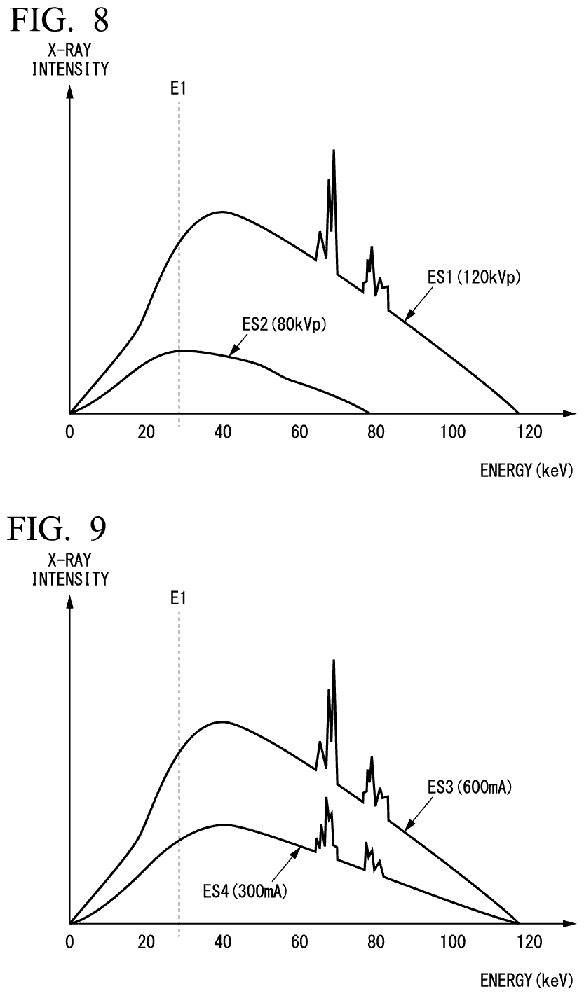

Next, the determination function 53-2 determines a correction coefficient (weighting coefficient) for each energy bin included in the determined range of energy bins in consideration of the energy spectrum of X-rays (step S303). FIG. 8 is a graph showing a relationship between the X-ray energy and the X-ray intensity for each tube voltage according to the second embodiment. As shown in FIG. 8, the intensity (energy spectrum ES1) of X-rays output from the X-ray tube 11 at a tube voltage of 120 kVp is generally higher than the intensity (energy spectrum ES2) of X-rays output from the X-ray tube 11 at a tube voltage of 80 kVp, but the difference in intensity varies depending on the value of energy. Specifically, the difference between the intensities increases as the value of energy increases. Therefore, a correction coefficient for reducing the influence of such a difference in the energy spectrum of X-rays (difference between the intensities) is determined for each energy bin. For example, for the energy band (20 to 40 keV) corresponding to the energy bin B2, the ratio of the X-ray intensity of the energy spectrum ES2 to the X-ray intensity of the energy spectrum ES1 at the average energy (30 keV) is determined as the correction coefficient. Reference data for determining such a correction coefficient is stored in the memory 41 as correction reference data D2. The correction coefficients may not be determined each time, but may be calculated in advance and stored as correction reference data D2 in the memory 41. Further, the determination function 53-2 may determine (adjust) the correction coefficients on the basis of an instruction input by an operator via the input interface 43.

Next, the determination function 53-2 determines reconstruction conditions including the determined range of energy bins and the correction coefficient for each energy bin (step S305). Thereafter, the generation function 53-3 reconstructs a photon counting CT image on the basis of the reconstruction conditions determined by the determination function 53-2 (using the determined count value and correction coefficient for each energy bin). For example, for energy bin B2, the generation function 53-3 does not use the count value as it is, but uses a value obtained by multiplying the count value by the correction coefficient (uses a reduced count value) to generate a photon counting CT image.

That is, the determination function 53-2 determines the correction coefficient on the basis of the difference between the energy spectrum of X-rays output from the X-ray tube at a tube voltage of the X-ray tube used at the time of capturing a past CT image and the energy spectrum of X-rays output from the X-ray tube at a tube voltage during new imaging using the photon counting CT apparatus, and the generation function 53-3 corrects count values included in the determined range of energy bins using the correction coefficient and generates a photon counting CT image using the corrected count values.

The intensity of X-rays (X-ray energy spectrum) also changes depending on the tube current of the X-ray tube 11. Therefore, when the tube current used at the time of capturing a past CT image is different from the tube current during new imaging, a correction coefficient may be determined in consideration of the influence of the energy spectrum caused by the difference in tube current. FIG. 9 is a graph showing a relationship between the energy of X-rays and the intensity of X-rays for each tube current according to the second embodiment. As shown in FIG. 9, the intensity (energy spectrum ES3) of X-rays output from the X-ray tube 11 at a tube current of 600 mA is generally higher than the intensity (energy spectrum ES4) of X-rays output from the X-ray tube 11 at a tube current of 300 mA, but the difference in intensity changes depending on the energy value. Therefore, a correction coefficient for reducing the influence of such a difference in the energy spectrum of X-rays (difference in the intensities) is determined. For example, for the energy band (20 to 40 keV) corresponding to the energy bin B2, the ratio of the X-ray intensity of the energy spectrum ES4 to the X-ray intensity of the energy spectrum ES3 at the average energy (30 keV) is determined as a correction coefficient. Here, if the tube current at the time of capturing a past CT image is “300 mA” and the tube current during new imaging is “600 mA,” the generation function 53-3 reconstructs a photon counting CT image on the basis of the reconstruction conditions determined by the determination function 53-2 (using the determined count value and the correction coefficient for each energy bin). For example, for the energy bin B2, the generation function 53-3 does not use the count value as it is, but generates a photon counting CT image using a value obtained by multiplying the count value by the correction coefficient calculated on the basis of the tube current (using a reduced count value).

That is, the determination function 53-2 determines a correction coefficient on the basis of the difference between the energy spectrum of X-rays passing through the subject and incident on the X-ray detector at the tube voltage of the X-ray tube used at the time of capturing a past CT image and the energy spectrum of X-rays incident on the X-ray detector at the tube voltage during new imaging using the photon counting CT apparatus, and the generation function 53-3 corrects the count values included in the determined range of energy bins using the correction coefficient, and generates a photon counting CT image using the corrected count values.

Instead of the spectrum of X-rays output from the X-ray tube 11, spectrum data of X-rays passing through the subject and incident on the X-ray detector 15 may be acquired in advance as the correction reference data D2. For example, the correction reference data D2 may be collected with a phantom placed on the bed device 30. The determination function 53-2 may determine a correction coefficient for each energy bin included in the determined range of energy bins using the correction reference data D2 of the energy spectrum of X-rays incident on the X-ray detector 15.

The tube current of the X-ray tube 11 may be adjusted to make the energy spectrum of X-rays output from the X-ray tube 11 or the energy spectrum of X-rays incident on the X-ray detector 15 as uniform as possible at different tube voltages.

According to the second embodiment described above, it is possible to compare medical images captured under different imaging conditions in a CT apparatus at the same energy level. Furthermore, by making such comparison of medical images possible, it is possible to support medical examinations by doctors. Moreover, since data in a wide energy range (e.g., 0 to 120 keV) can be obtained in new imaging using the photon counting CT apparatus, it is also possible to generate a spectral image unique to the photon counting CT apparatus. Furthermore, by reconstructing a photon counting CT image in consideration of the energy spectrum of X-rays output from the X-ray tube or the energy spectrum of X-rays incident on the X-ray detector, it is possible to compare medical images under more uniform imaging conditions.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical image processing apparatus comprising processing circuitry configured to:
acquire imaging conditions including a tube voltage of an X-ray tube used at a time of capturing a past CT image of a subject;
determine a range of energy bins to be used to generate a photon counting CT image corresponding to the past CT image among a plurality of energy bins set for counting X-ray photons detected by new imaging of the subject using a photon counting CT apparatus for each energy band based on the acquired imaging conditions; and
generate the photon counting CT image using count values of the X-ray photons in the determined range of energy bins.

2. The medical image processing apparatus according to claim 1, wherein the processing circuitry is configured to determine, as the range of energy bins, a range of energy bins from an energy bin having a lowest energy band among the plurality of energy bins to an energy bin having an energy band corresponding to a value of the tube voltage included in the imaging conditions.

3. The medical image processing apparatus according to claim 1, wherein the processing circuitry is configured to determine, as the range of energy bins, a range of energy bins from an energy bin having a lowest energy band among the plurality of energy bins to a boundary value between energy bins closest to a value of the tube voltage included in the imaging conditions.

4. The medical image processing apparatus according to claim 1, wherein the processing circuitry is configured to:
determine, as the range of energy bins, a range of energy bins from an energy bin having a lowest energy band among the plurality of energy bins to an upper energy bin whose energy band includes a value of the tube voltage included in the imaging conditions; and
with respect to count values of the upper energy bin among the count values in the determined range of the energy bins, adjust count values used to generate the photon counting CT image based on a difference between a lower limit value of the energy band of the upper energy bin and the value of the tube voltage included in the imaging conditions.

5. The medical image processing apparatus according to claim 1, wherein the processing circuitry is configured to:
determine a correction coefficient based on a difference between an energy spectrum of X-rays output from the X-ray tube at the tube voltage of the X-ray tube used at the time of capturing the past CT image and an energy spectrum of X-rays output from the X-ray tube at a tube voltage during the new imaging using the photon counting CT apparatus;
correct the count values included in the determined range of energy bins using the correction coefficient; and
generate the photon counting CT image using the corrected count values.

6. The medical image processing apparatus according to claim 1, wherein the processing circuitry is configured to:
determine a correction coefficient based on a difference between an energy spectrum of X-rays passing through the subject and incident on an X-ray detector at a tube voltage of the X-ray tube used at the time of capturing the past CT image and an energy spectrum of X-rays incident on the X-ray detector at a tube voltage during the new imaging using the photon counting CT apparatus,
correct the count values included in the determined range of energy bins using the correction coefficient, and
generate the photon counting CT image using the corrected count values.

7. The medical image processing apparatus according to claim 1, wherein the processing circuitry is configured to cause a display device to display both the generated photon counting CT image and the past CT image such that both CT images are able to be compared.

8. A medical image processing method, using a computer, comprising:
acquiring imaging conditions including a tube voltage of an X-ray tube used at a time of capturing a past CT image of a subject;
determining a range of energy bins to be used to generate a photon counting CT image corresponding to the past CT image among a plurality of energy bins set for counting X-ray photons detected by new imaging of the subject using a photon counting CT apparatus for each energy band based on the acquired imaging conditions; and
generating the photon counting CT image using count values of the X-ray photons in the determined range of energy bins.
